(19) United States
(12) Patent Application Publication
(10) Pub. No.: US 2012/0101509 A1
(43) Pub. Date: Apr. 26, 2012

(54) SURGICAL RING WITH ASSEMBLED CONSTRUCTION

(75) Inventor: Pascal Paganon, Lyon (FR)

(73) Assignee: Compagnie Europeenne d'Etude et de Recherche de Dispositifs pour l'Implantation par Laparoscopie, Vienne (FR)

(21) Appl. No.: 13/276,711

(22) Filed: Oct. 19, 2011

Related U.S. Application Data
(63) Continuation of application No. PCT/EP2010/054916, filed on Apr. 15, 2010.

(30) Foreign Application Priority Data
Apr. 21, 2009 (FR) 0952607

(51) Int. Cl. A61F 2/04

(52) U.S. Cl. 606/151

(57) ABSTRACT

A surgical ring is suitable for implantation in a human or animal body and to surround a biological organ forming a bag in order to modify the flow area thereof. The ring comprises a hose having an inner surface intended for engaging with the biological organ in order to modify the section thereof and an outer surface opposite the former, and a belt mounted on and along the outer surface and attached to the latter. The hose and the belt are respectively provided with a first and second matching assembly that interact mechanically in order to substantially prevent any sliding of the belt along the outer surface.

The present invention pertains to the general technical field of surgical implants to be implanted in a patient's body around a biological organ or organs, so as to constitute a pouch or a conduit, and more particularly to gastric rings designed to treat obesity by the implantation of a flexible gastric ring designed to form a closed loop around the stomach or the esophagus to reduce its section of passage.

The present invention pertains to a surgical ring implantable in a human or animal body to surround a biological organ constituting a pouch or a conduit in order to modify the section of passage of said organ, said ring comprising firstly a tube or hose having an internal face designed to come into contact with a biological organ in order to modify its section and an opposite external face, and secondly a belt placed against and along said external face and attached to this external face.

The present invention pertains more particularly to a gastric ring but it can also pertain to a ring designed to treat urinary or fecal incontinence (such as an artificial sphincter) or again a ring designed to adjust the blood flow in blood vessels, this list being non-exhaustive.

There are already known ways of carrying out surgical operations on patients afflicted with extremely severe obesity (or morbid obesity), i.e. patients whose weight exceeds their ideal weight by at least 50 kg, through the implanting of a gastric ring (also called a gastroplasty ring) into these patients' bodies.

This widely known technique consists of the implanting of a gastric ring around the patient's stomach in order to reduce the size of the stomach as well as the diameter of its passage (the stoma).

In particular, there is a known gastric ring comprising firstly a flexible inflatable hose and secondly a dorsal belt attached to and fixed along the inflatable hose. The ring is closed by means of an appropriate locking device positioned at the ends of the belt and enabling the ring to take the form of a closed loop circularly gripping the stomach. The volume of the inflatable hose is adjusted by the addition or removal of an inflating fluid. To this end, the inflatable hose is connected to one of the ends of a catheter, the other end of which is itself connected to a subcutaneous implantable site enabling the introduction or removal of inflating fluid through the patient's skin, by means of a hollow needle piercing the impervious membrane of the site in question. The main function of the dorsal belt is to contain the deformation of the inflatable hose so as to favor a primarily centripetal inflation of this hose towards the stomach.

This construction of a ring by the association of two distinct and independent components (namely the hose and the belt) has many worthwhile features, as well as certain drawbacks, explained here below.

First of all, this prior art ring proves to be relatively difficult to manufacture. Indeed, the belt is fixed on and along the hose by gluing done manually by an operator. The relative positioning of the belt with respect to the hose during the gluing must be extremely precise in order to obtain a perfectly functional and non-traumatic ring.

Such precision however is difficult to attain in practice since the operator may inadvertently glue the belt along the hose with a longitudinal offset from the ideal position determined by the design of the ring of the position of the belt relatively to the hose. This risk of a longitudinally offset fastening of the belt relatively to the hose is increased by the fact that it is difficult for the operator, during manufacture, to swiftly and precisely identify the ideal positioning of the belt relatively to the hose. For the above reasons, the manufacture of prior art rings designed on the basis of the association of a hose and a belt proves to be difficult, lengthy and costly with a reject rate that may be significant and that contributes to increasing the cost of these rings.

Furthermore, there is a risk of deterioration of the layer of glue that bonds together the inflatable hose and the belt, and this could lead to an accidental separation of the belt from the hose, with all the negative consequences that this would have especially for the patient's safety.

The document EP1829505 describes a gastric ring extender. The extender gets fixed to a first end of a gastric ring. The gastric ring has latching means at both ends. The extender has an elongated body, one end of which gets coupled to a latching means for the gastric ring.

The objects assigned to the present invention are therefore aimed at remedying the different drawbacks mentioned here above and at proposing a novel surgical ring that can be manufactured easily quickly and at low cost while at the same time being particularly reliable in its use.

Another object assigned to the invention is aimed at proposing a novel surgical ring that is particularly reliable and robust in its construction. Another object of the invention is aimed at proposing a novel surgical ring of a construction that is extremely simple and easy to achieve.

Another object of the invention is aimed at proposing a novel surgical ring with a gripping diameter that can be adjusted, and with a design that is particularly compact, implementing a minimum number of separate parts.

Another object of the invention is aimed at proposing a novel surgical ring with a particularly resistant structure.

The objects assigned to the invention are attained by means of a surgical ring implantable in a human or animal body to surround a biological organ constituting a pouch or a conduit in order to modify the section of passage of said organ, said ring comprising firstly a hose having an inner face designed to come into contact with the biological organ to modify its section and an opposite external face, and secondly a belt placed against and along said external face and attached to this external face, characterized in that the hose and the belt are respectively provided with first and second complementary assembling means which interact mechanically to substantially prevent any slipping of the belt along the external face.

Other objects, features and advantages of the invention shall appear in greater detail from the reading of the following description, as well as from the appended drawings, given purely by way of a non-exhaustive illustration. Of these figures:

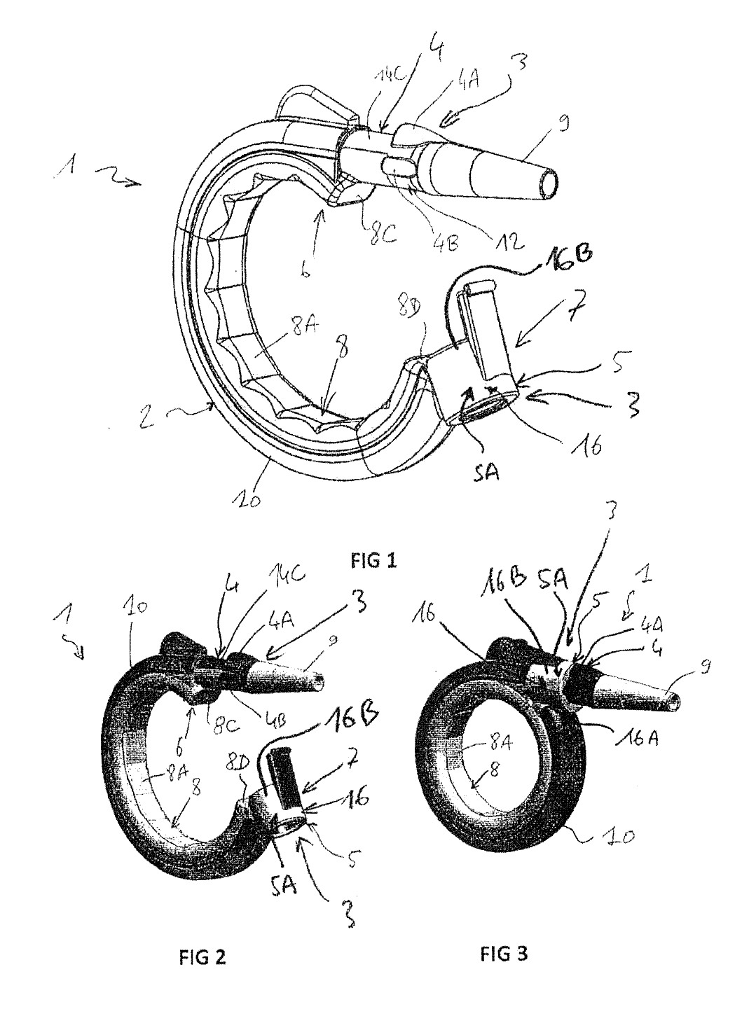

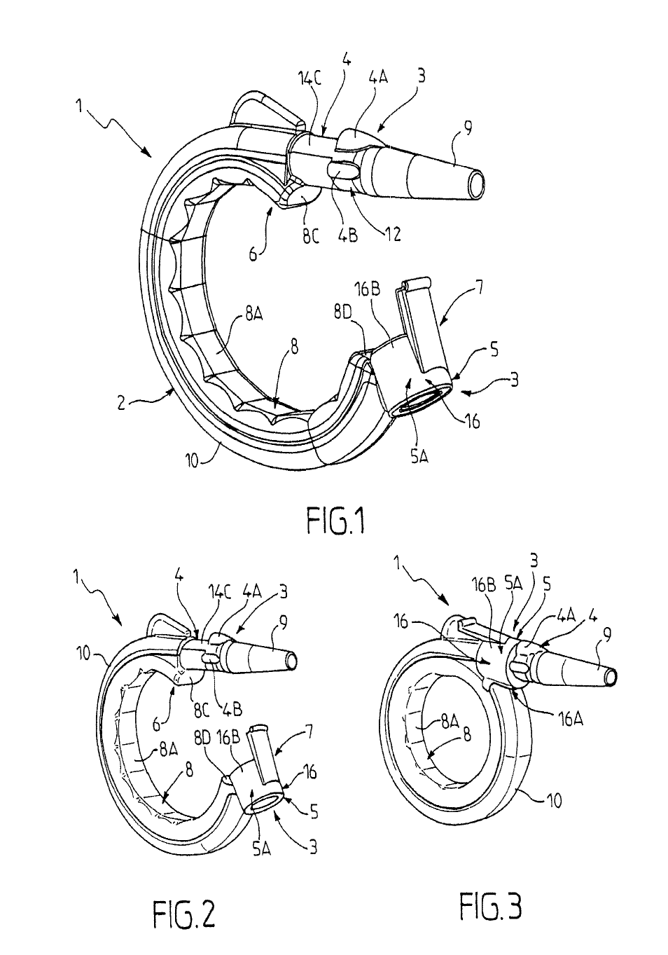

FIG. 1 is an illustration, in a schematic view in perspective, of a first embodiment of a surgical ring for treating obesity designed to grip the stomach or the esophagus, said ring being in an unlocked configuration.

FIG. 2 is an illustration, in a schematic view in perspective, of a ring according to a second embodiment of the invention, this ring being distinguished from the ring of FIG. 1 only by the fact that its belt and its hose are made out of materials of different colors, the color of the belt being thus darker than that of the hose, said ring being in an unlocked configuration like that of FIG. 1.

FIG. 3 is an illustration, in a schematic view in perspective of the ring of FIG. 2 in its locked configuration.

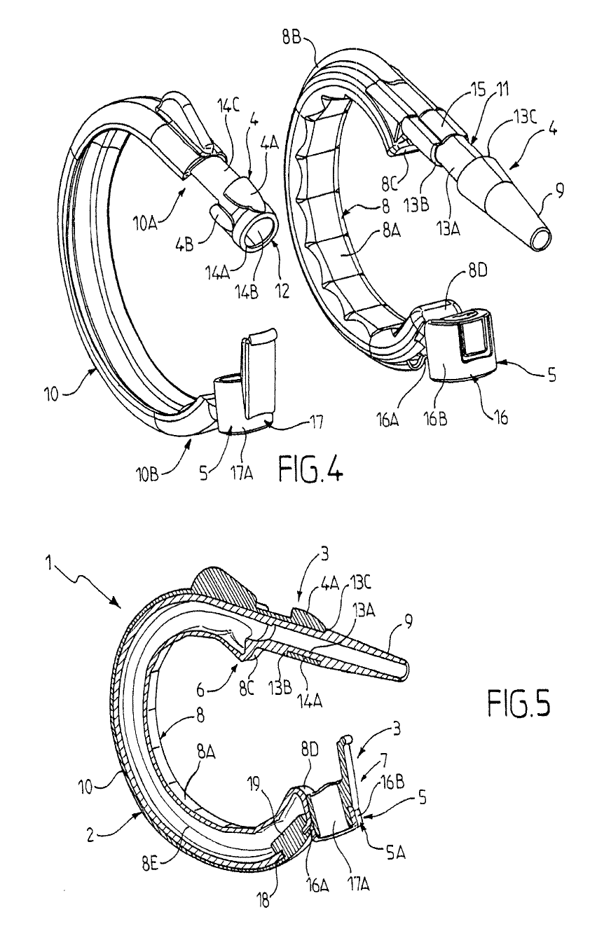

FIG. 4 is an exploded view of the ring of FIG. 1 in which the hose and the belt are separated to enable a more precise understanding of their respective structures.

FIG. 5 is a schematic view in section of the ring of FIG. 2.

Here below in the description, we shall refer to a surgical ring 1 for treating obesity, generally called a “gastric ring” and designed to surround the stomach or the esophagus so as to reduce the section of passage thereof.

However, the invention is not necessarily limited to this application and may also relate to other surgical rings and generally to surgical rings designed to be implanted in a patient's body around at least one hollow biological organ constituting for example a pouch or a conduit to modify the section of passage of said organ where it is gripped by the ring. For example, we may cite the rings used to treat urinary or fecal incontinence or again those used around blood vessels to adjust the blood flow. In the case of the treatment of urinary incontinence, the ring will be implanted around the bladder or the urinary tract, and in the case of fecal incontinence, it will be implanted around the gastro-intestinal tract and especially around the anal structures of the intestine.

As explained here above, the surgical ring 1 is a gastric ring for the surgical treatment of obesity, i.e. the ring 1 is designed to be surgically implanted inside the patient's body, around the stomach or esophagus (depending on the surgical technique chosen) to locally constrict the stomach or esophagus and thus locally reduce the section of passage of food in the stomach or esophagus. This restriction of section slows down the flow of food, enabling the patient to arrive at a sensation of fullness fairly quickly and with a relatively small quantity of food.

For example, the ring 1 according to the embodiments illustrated in the figures can be designed to implement an adjustable-ring gastroplasty treatment, sometimes called annuloplasty or again the adjustable gastric banding (AGB) procedure.

This ring 1 can also, alternatively, be designed for a treatment known as the “adjustable bypass” treatment in which the ring 1 is positioned around a gastric pouch resulting from a surgical stomach bypass operation. Preferably, the ring 1 is designed to be implanted by laparoscopy, i.e. it has dimensions and properties which make it capable of being inserted, by means of a trocar, into the patient's body by a surgical operation known as the “closed stomach” operation.

The surgical ring 1 includes for example a flexible band 2, preferably made out of an elastomer, such as silicone and especially medical-grade silicone.

The flexible band 2 is designed to be closed about the chosen biological organ (in this case the esophagus or the stomach) in a predetermined locking configuration (illustrated in FIG. 3) in which it takes the form of a closed loop, so as to locally grip the organ on a perimeter whose length is advantageously adjustable, through optional technical means which shall be described in greater detail here below. In order to obtain this closing of the flexible band 2 on itself, which corresponds to the locking configuration illustrated in FIG. 3, the ring 1 advantageously has means 3 for locking in a closed functional position around the biological organ to be gripped. Preferably, the locking means 3 include male and female locking elements 4, 5 positioned respectively towards the first end 6 and a second end 7 of the ring 1, i.e. specifically the ends of the flexible band 2 forming the ring 1. For example, as shown in the Figures, the male locking element 4 advantageously includes an elongated locking element designed to be threaded by force into a collar 5A forming the female element 5, said elongated locking element being provided with stops 4A, 4B preventing the untimely reopening of the band 2 once it has been closed and locked around the stomach but enabling an unlocking and a reopening of the band 2 provided sufficient force is applied and directed in an appropriate direction to elastically deform the stops 4A, 4B and/or the collar 5A forming the female element 5 to enable the disengagement of the male and female elements 4, 5. The band 2 can thus advantageously pass as desired and reversibly from an open configuration (illustrated in FIGS. 1, 2 and 4) to a predetermined closing configuration (illustrated in FIG. 3) in which the male and female locking elements 4, 5 cooperate in a fitted-in locking position so as to keep the ends 6, 7 of the flexible band 2 joined.

As illustrated in the Figures, the ring 1 has a hose 8 presenting an inner face 8A designed to come into contact with the biological organ to modify its section of passage and an opposite external face 8B. The hose 8 advantageously has an elongated shape, i.e. extending longitudinally between a first end 8C and a second end 8D. The hose 8 advantageously has the form of a hollow flexible tube extending between two closed ends corresponding to the first and second ends 8C, 8D, said closed ends being made to abut each other in the predetermined locking configuration of the band 2 (cf. FIG. 3) so as to form a substantially annular hose 8 capable of gripping the biological organ on an angular range substantially equal to 360°. In this case, the hose 8 thus stretches longitudinally in the major part of the length of the flexible band 2 and preferably extends substantially over the totality of the length of the gripping perimeter formed the band 2 when it is in its predetermined locking configuration (cf. FIG. 3).

As explained here above, the hose 8 is hollow, i.e. it demarcates an internal closed volume or in other words a closed chamber 8E.

Advantageously, the hose 8 is designed to contain a filler fluid which is preferably constituted by a liquid of low viscosity, such as for example a physiological liquid or a saline solution. However, it is quite possible to envisage a case where the filler fluid of the hose 8 consists of a gas such as air or conversely a paste or semi-paste, without in any way departing from the framework of the invention.

Thus, by adding or withdrawing fluid to or from the hose 8 (and more specifically into or from chamber 8E), it is possible to adjust the gripping diameter defined by the internal face 8A. To this end, the ring 1 advantageously has a means for making the interior of the hose 8 communicate with the exterior to enable an input of inflating fluid into the hose 8 or on the contrary to enable the withdrawal of inflating fluid out of the hose 8. For example, the means used to make the interior of the hose 8 communicate with the exterior include an aperture made in the hose 8, said aperture being connected to a nozzle 9. The nozzle 9 is to be linked to a catheter which is itself connected to a box (an implantable site) designed to be implanted beneath the patient's skin to enable the injection (or withdrawal) of fluid into or from the hose 8 by means of the catheter and the nozzle.

However, it is quite possible, without departing from the framework of the invention, to envisage a case where the hose 8 is not to be filled with fluid but on the contrary has to receive a solid reinforcing body (for example a metal wire) constituting a core to prevent any diametrical deformation of the ring 1 under the effect of the centrifugal expansion force exerted by the gripped biological organ. In this example (which does not correspond to the variants illustrated), the ring 1 does not have any adjustable character.

The ring 1 furthermore comprises a belt 10 formed by a part that is substantially distinct and independent of the hose 8 and is placed against and along the external face 8B of the hose 8 and attached to this hose 8. In other words, the belt 10 advantageously has an elongated shape with a length comparable to that of the hose 8 and designed to be fixedly joined against and along the hose 8 so as to form an indissociable unit with this hose 8, thus constituting the ring 1.

Thus, the ring 1 is advantageously constituted by the permanent and definitive association of two one-piece parts, namely on the one hand a hose 8 which by itself preferably forms an inflation chamber and on the other hand a belt 10 which advantageously takes the form of an elastomer strip designed to be placed to and fixed against and along the hose 8. The belt 10 is advantageously made out of an elastomer material, preferably silicone. Preferably, the material constituting the belt 10 has a hardness greater than that of the material constituting the hose 8, so as to give the belt 10 sufficient mechanical strength to enable it to limit the centrifugal radial expansion of the ring 1 during the inflation of the hose 8. On the contrary, the material constituting the hose 8 is chosen to have a particularly soft and flexible character so as not to damage the biological tissues.

The hose 8 and the belt 9 are respectively provided with complementary assembling means 11, 12 which mechanically interact to substantially prevent any slipping of the belt 10 along the external face 8B of the hose 8. In other words, the first assembling means 11 which are fixedly joined to the hose 8 and the second assembling means 12 which are fixedly joined to the belt 10 cooperate mechanically, i.e. by mutual blocking contact so as to prevent any relative longitudinal shift of the belt 10 and of the hose 8 when the belt 10 is placed in a functional position against the hose 8. Through this technical step, the position of the belt 10 relatively to the hose 8 is identified and fixed reliably by mechanical inter-locking (i.e. without any glue or solder) without any risk of introducing an untimely longitudinal offset of the belt 10 relatively to the hose 8 by relative longitudinal slipping of the belt 10 and of the hose 8.

As explained here above, the first and second assembling means 11, 12 cooperate so as to fixedly join the belt 10 and the hose 8 together in a purely mechanical way, i.e. without gluing, soldering or other means of joining that are not related to a mechanical interaction proper.

The invention thus relies on the implementation of means 11, 12 which mechanically fix the longitudinal position of the belt 10 relatively to the hose 8, i.e. which prevent at least one relative longitudinal sliding of the hose 8 and the belt 10.

Advantageously, the first and second complementary assembling means 11, 12 interact mechanically to attach the belt 10 to the hose 8, i.e. to fixedly join, at least locally, the belt 10 to the hose 8. In this example, which corresponds to the variants illustrated in the figures, the mechanical link achieved by interaction between the first and second assembling means 11, 12 is an embedded type of link preventing the dissociation of the hose 8 and the belt 10.

Advantageously, the first and second assembling means 11, 12 comprise at least one first male fastening element 13 and one first female fastening element 12 mechanically coupled together.

In other words, the mechanical interaction of the first and second assembling means 11, 12 is in this case obtained by the fitting together of a first male fastening element 13 into a first female fastening element 14, the first male fastening element 13 and the first female fastening element 14 being mechanically interlocked, thus achieving the fastening, at least locally, of the belt to the hose 8 in a predetermined relative position.

Preferably, the first assembling means 11, with which the hose 8 is provided, comprise the first male fastening element 13 while the second assembling means 12, with which the belt 10 is provided, comprise the first female fastening element 14. Preferably, the first female fastening element 14 itself comprises a fastening collar 14A while the first male fastening element 13 comprises a fastening segment 13A which stretches between two fastening stops 13B, 13C, said fastening segment 13A being inserted into the fastening collar 14A in such a way that this collar is blocked between said fastening stops 13B, 13C thus immobilizing the hose 8 relatively to the belt 10 in a unique predetermined position. Preferably, the fastening collar 14A demarcates a through hole 14B with a substantially circular section, the fastening segment 13A itself having a tubular shape which is substantially complementary to that of the hole defined by the fastening collar 14A so as to obtain an adjusted assembling of the first female fastening element 14 and the first male fastening element 13.

Advantageously, the first male fastening element 13 incorporates means for setting up communication between the interior of the hose 8 and the exterior, referred to here above. More specifically, in the context of the variants illustrated in the Figures, the fastening segment 13A is hollow so as to form a conduit for making the interior of the hose 8 communicate with the exterior, the conduit in question being prolonged by the nozzle 9 which extends longitudinally in the continuity of the first male fastening element 13. This technical step reduces the number of constituent parts of the ring 1 and thus gives this ring 1a character that makes it particularly compact, robust and easy to manufacture.

Advantageously, the first and second complementary assembling means 11, 12 are positioned towards a first end 8C, 10A, respectively of the hose 8 and of the belt 10 so as to set up a mechanical anchoring point for anchoring the belt 10 to the hose 8 towards a first end 6 of the flexible band 2.

Advantageously, the first and second complementary assembling means 11, 12 contribute to forming the male locking element 4. Thus, in the embodiment shown in the Figures, the fastening collar 14a carries locking stops 4A, 4B on its periphery, said collar 14a being situated at the first end 10A of the belt 10 and being connected to the rest of said belt 10 by means of a joining portion 14C that matches a complementary zone 15 made on the hose 8 to form the male locking element. This technical step, in which the first and second complementary assembling means 11, 12 are made to at least partly form the male locking element 4, ensures the fixed joining of the belt 10 to the hose 8 since, during the locking of the ring 1, the collar 5A forming the female locking element 5 exerts a mechanical stress on the male element 4 which tends to maintain the joining of the first and second assembling elements 11, 12.

Advantageously, the hose 8 and the belt 10 are respectively provided with third and fourth complementary assembling means 16, 17 which mechanically interact to substantially prevent any slippage of the belt 10 along the external face 8B. In other words, the third and fourth assembling means 16, 17 fulfill a function that is identical and therefore redundant to the one provided by the first and second assembling means 11, 12 described here above. This redundancy makes it possible however to further reinforce the structure of the ring 1 while at the same time constituting an additional foolproofing element for the mounting of the belt 10 on the hose 8.

Preferably, the third and fourth complementary assembling means 16, 17 are positioned towards a second end 8D, 10B respectively of the hose 8 and the belt 10. The variants illustrated in the figures thus implement two mechanical fastening points between the belt 10 and the hose 8 respectively positioned at each of the ends of these elements, i.e. towards each of the ends 6, 7 of the flexible band 2.

Advantageously, the third and fourth complementary assembling means 16, 17 contribute to forming the female locking element 5. For example, as illustrated in the Figures, the fourth assembling element 17 includes a first fastening collar 17A designed to be inserted inside a second fastening collar 16B concentrically with respect to this latter collar, the association of said first and second fastening collars 17A, 16B forming the locking collar 5A. More specifically, the first fastening collar 17A is inserted by force into the aperture demarcated by the second fastening collar 16B by means of a notch 16A made in the side wall of said second fastening collar 16B.

Advantageously, the ring 1 can also, as can be seen more particularly in FIG. 5, include fifth and sixth complementary assembling means 18, 19 that mechanically interact to substantially prevent any sliding of the belt 10 along the external face 8B.

The fifth and sixth assembling means 18, 19 are therefore redundant relatively to the first, second, third and fourth assembling means 11, 12, 16, 17 referred to here above.

For example, the fifth assembling means 18 consist of an aperture made in the wall of the hose 8 while the sixth assembling means 19 consist of a mushroom-shaped protrusion emerging from of the belt 10, the apertures and the protrusions in question cooperating in a manner of a button and a button-hole, the button being formed by the protrusion while the button-hole is formed by the aperture. Advantageously, the aperture forming a fifth assembling means 18 may correspond to the aperture made necessary by the method of manufacturing the hose 8 by molding. Indeed, when the hose 8 is manufactured by molding, it is necessary to be able to remove the core of the mold through an aperture which may correspond to the aperture forming the fifth assembling means 18. This aperture must naturally be plugged so that the core 8 demarcates an impervious chamber 8E capable of receiving an inflation fluid. The aperture in question may thus be plugged through the mushroom-shaped protrusion which, in this case, fulfills a dual role of providing imperviousness and a mechanical link.

Advantageously, the belt 10 is glued to the hose 8. Preferably, all the surfaces of the belts 10 and the hose 8 designed to come into contact are glued together so as to enable a close, uniform and continuous joining of the belt 10 and of the hose 8 throughout their contact interface.

This gluing advantageously complements the mechanical joining obtained by at least the first and second assembling means 11, 12.

The simultaneous implementation of mechanical assembling by means of said at least first and second assembling means 11, 12 and a gluing of the belt 10 and of the hose 8 throughout their contact interface makes it possible to obtain a ring 1 of a particularly robust and lasting construction.

An example of the manufacture and use of a ring 1 according to the invention shall be described here below.

First of all, the hose 8 is manufactured by an operation for molding an elastomer material, which gives a single-piece unit forming one part with said hose 8.

Similarly, the belt 10 is manufactured by an operation for molding elastomer material (preferably with a hardness greater than that of the material forming the hose) giving an integral part forming the belt 10 in question.

Then, a manufacturing operator implements the following operations:
A first operation for fixedly joining the belt 10 to the hose 8 by making the hose 8 enter the aperture 14B by the nozzle 9 until the fastening segment 13A is inserted into the aperture 14B and the collar 14A is blocked in longitudinal translation between the stops 13B, 13C.
A second operation for mechanically joining the belt 10 to the hose 8 made as follows: at the opposite end 7 of the flexible band 2, the collar 17A is inserted by force through the slot 16A into the second collar 16B until it is substantially coaxial with this collar 16B.
A third operation for mechanically joining the belt 10 to the hose 8 made as follows: the protrusion forming the sixth assembling means 19 is inserted by force into the aperture forming the fifth assembling means 18 in the manner of a button in a button hole.

These different mechanical assembling operations enable the position of the belt 10 to be fixed very precisely relatively to the hose 8. These mechanical assembling operations are complemented by a gluing of the assembling means in sets of two (the first assembling means 11 are glued to the second assembling means 12, the third assembling means 16 are glued to the fourth assembling means 17 and the fifth assembling means 18 are glued to the sixth assembling means 19).

The operator also performs a uniform gluing of the hose 8 and the belt 10, this gluing being done by coating the totality of the surfaces of the hose 8 and the belt 10 that are to come into contact with glue. The glue is introduced between the hose 8 and the belt 10 after these two elements have been mechanically assembled, by means of the following procedure:
the contact surfaces of the hose 8 and of the belt 10 are moved away from each other.
then the precise quantity of glue needed is inserted at the contact interface,
finally the contact surfaces are released and come into contact naturally by elastic return; the contact surfaces of the hose 8 and of the belt 10 are naturally and automatically pressed against one another because of the longitudinal tension of the belt 10 on the core 8 (imparted by mechanical assembling), thus favoring the gluing.

The implementation of the gluing is thus very simple and perfectly reproducible.

During use, the ring 1 is locked by the introduction of the male element 4 into the female element 5 (constituted by the unit sub-assembly formed by the first and second coaxial collars 17A, 16B glued to each other), thus reinforcing the assembling of the first and second assembling means 11, 12 on the one hand and the third and fourth assembling means 16, 17 on the other hand, the collar 17A cooperating especially with the stops 4A, 4B while the male element 4 is reciprocally inserted into the collar 17A, thus keeping this collar 17A in position relatively to the hose 8.

1. Surgical ring implantable in a human or animal body to surround a biological organ constituting a pouch or a conduit in order to modify the section of passage of said organ, said ring comprising
a hose having an inner face designed to come into contact with the biological organ to modify its section, and an opposite external face, and
a belt placed against and along said external face and attached to this external face,
wherein the hose and the belt are respectively provided with first and second complementary assembling members which interact mechanically to substantially prevent any slipping of the belt along the external face.

2. Ring according to claim 1 wherein said first and second complementary assembling members interact mechanically to attach the belt to the hose.

3. Ring according to claim 1, wherein the first and second assembling members comprise at least one first male fastening element and one first female fastening element mechanically coupled together.

4. Ring according to claim 3, wherein the first assembling member comprises said first male fastening element while said second assembling member comprises said first female fastening element.

5. Ring according to claim 1, wherein the hose is designed to contain a filler fluid.

6. Ring according to the claim 4 wherein said first male fastening element incorporates a structure for making the interior of the hose communicate with the exterior to enable an input of inflating fluid into the hose.

7. Ring according to claim 3 wherein said first female fastening element comprises a fastening collar while the first male fastening element comprises a fastening segment which stretches between two fastening stops, said fastening segment being inserted into the fastening collar in such a way that this the collar is blocked between said fastening stops.

8. Ring according to claim 1, wherein the hose and the belt are respectively provided with third and fourth complementary assembling members which mechanically interact to substantially prevent any slippage of the belt along the external face.

9. Ring according to claim 8, wherein said first and second complementary assembling members are positioned towards a first end, respectively of the hose and of the belt, and said third and fourth complementary assembling members being are positioned towards a second end respectively of the hose and the belt.

10. Ring according to claim 1, wherein the ring further comprises a locking member for locking into a closed functional position around the biological organ to be gripped.

11. Ring according to claim 10 wherein said locking member includes male and female locking elements positioned respectively towards a first end and a second end of the ring.
